(19) United States
(12) Patent Application Publication
(10) Pub. No.: US 2019/0307587 A1
(43) Pub. Date: Oct. 10, 2019

(54) STRUCTURE FOR A CATHETER SLEEVE AND CATHETER SLEEVE

(71) Applicant: BIOTRONIK AG, BUELACH (CH)

(72) Inventor: AMIR FARGAHI, BUELACH (CH)

(21) Appl. No.: 16/375,153

(22) Filed: Apr. 4, 2019

(30) Foreign Application Priority Data
Apr. 4, 2018 (EP) 18165617.4

(51) Int. Cl. A61F 2/966; A61F 2/24

(52) U.S. Cl. CPC A61F 2/966; A61F 2/2436; A61M 25/0668; A61F 2002/9665; A61F 2/2418

(57) ABSTRACT

A structure for a catheter sleeve is characterized by a particularly high flexural elasticity. The tubular structure has at least one S-shaped profiled strip which is arranged along the structure in a helix. The at least one S-shaped profiled strip has a U-shaped longitudinal section in the region of each side edge. A respective U-shaped longitudinal section of the profiled strip engages with an opposing, U-shaped longitudinal section of a directly adjoining turn of the helix of the same profiled strip or of a further S-shaped profiled strip adjoining in the helix. The structure may further be used in a corresponding catheter.

### CROSS-REFERENCE TO RELATED APPLICATION

This application claims the priority, under 35 U.S.C. § 119, of European application EP 18165617.4, filed Apr. 4, 2018; the prior application is herewith incorporated by reference in its entirety.

### BACKGROUND OF THE INVENTION

### Field of the Invention

The present application relates to a tubular structure for a catheter sleeve and to a corresponding catheter sleeve and to a corresponding catheter.

Medical implants, in particular intraluminal endoprostheses, for a wide variety of applications are known from the state of the art in great diversity. Implants are endovascular prostheses or other endoprostheses, for example, such as stents (stents for vessels, (vascular stents, including stents for use in the area of the heart and heart valve stents, such as mitral valve stents, pulmonary valve stents) and bile duct stents), endoprostheses for closing patent foramen ovale (PFO), stent grafts for treating aneurysms, endoprostheses for closing an atrial septal defect (ASD), and prostheses in the area of hard and soft tissues.

Such an implant usually assumes two states, namely a compressed state having a small diameter and an expanded state having a larger diameter. In the compressed state, the implant can be inserted into the vessel or organ to be treated through narrow vessels by a catheter and positioned at the site to be treated. Fixation in the compressed state frequently takes place by a catheter sleeve (also referred to as a capsule), which is arranged at the distal end of the catheter. In the expanded state, the implant remains in the vessel or organ and is secured there after the catheter has been removed from the body of the treated patient. In the case of a transcatheter aortic valve implantation (TAVI, endovascular aortic valve replacement), for example, an artificial aortic valve is introduced into the heart in a tubular scaffold, for the implantation of which today primarily catheters made of plastic materials or composites are used, which have limited pliability and flexibility. The valve is brought into position by catheters. Afterwards, the valve is unfolded and anchored. The endogenous aortic valve is not removed, but displaced by the implant. In the case of a self-expanding implant made of a shape memory alloy, the implant transitions into the expanded state when a transformation temperature is exceeded or mechanical stress exerted on the implant is no longer present.

During the insertion of the implant by the catheter, the catheter sleeve is guided or positioned along blood vessels. The catheter sleeve conforms to the respective shape of the inner volume of the vessel in the process and is exposed to a changing bending force or changing compressive and tensile stress in the process. In particular the passage of a catheter through the aortic arch results in severe deformation of the catheter sleeve. The deformation of the catheter sleeve can impair the function of the catheter and/or of the implant arranged in the catheter sleeve.

Examples of catheter sleeves according to the prior art can be derived from the publications international patent disclosure WO 2011/133368 A1 (corresponding to U.S. Pat. Nos. 8,465,541 and 9,492,275), published, European patent application EP 1 723 937 A1 (corresponding to U.S. patent publication No. 2006/9259121) and U.S. patent publication No. 2011/0098804 A1. U.S. patent publication No. 2011/0098804 A1 discloses a catheter sleeve comprising, in the central section, a plurality of helical rings, which are separated from one another by appropriate notches. The notches extend in the circumferential direction across less than 180°, so that the rings are connected by one or more ribs extending in the longitudinal direction. A similar shape of a catheter sleeve (catheter tube) is also disclosed in the published, European patent application EP 1 723 937 A1. International patent publication WO 2011/133368 A1 shows a catheter sleeve including slits extending in the longitudinal direction.

### SUMMARY OF THE INVENTION

It is thus the object of the present invention to provide a structure for a catheter sleeve which has greater flexural elasticity and/or a greater resistance to compressive or tensile stress during implantation. Accordingly, it is the object to impart greater flexural elasticity to the sleeve of a catheter.

The object is achieved by the structure having the features of the independent claim.

The tubular (hollow-cylindrical) or sleeve-shaped structure according to the invention comprises, in particular, at least one S-shaped profiled strip, which is arranged along the structure in a helix, wherein the at least one S-shaped profiled strip has a U-shaped longitudinal section in the region of each side edge. A respective U-shaped longitudinal section of the profiled strip engages with an opposing, U-shaped longitudinal section of a directly adjoining turn of the helix of the same profiled strip or of a further S-shaped profiled strip adjoining in the helix. In the second case, the structure thus comprises at least two mutually engaging profiled strips, which each extend in a helix. The at least two profiled strips form a multi-turn helix. In the first case, the helix is formed by only a single profiled strip.

The tubular structure particularly forms an inside diameter in the range between 3.7 and 5.7 mm, and preferably between 4.4 mm and 5 mm. The outside diameter is particularly 6 mm or smaller, preferably 5.3 mm or smaller, particularly preferably 4.7 mm or smaller, and in particular the outside diameter is preferably 4 mm or smaller. However, it is clear to a person skilled in the art that the inside diameter in any case is smaller than the outside diameter.

The S-shaped profiled strip is, or the S-shaped profiled strips are, arranged or wound along a helical line or a coil or spiral (helix) so as to extend with a constant slope along and around the jacket of the tubular structure. Within one convolution over 360°, the respective helix covers the so-called pitch, which extends parallel to the longitudinal axis of the helix. As will be described in greater detail hereafter, the profiled strip engages, or the profiled strips engage one another, whereby a complete tube or hollow cylinder, this being the structure according to the invention, is formed, which does not include any free spaces (such as slits) between the profiled strips. The hollow cylinder shape is completely filled or covered by the profiled strip or the profiled strips arranged in the helix. When multiple profiled strips are used, these are arranged next to one another along the circumference of the helix. The helix described by the profiled strip or the profiled strips can be right-handed (i.e. wound clockwise) or left-handed (i.e. wound counterclockwise).

Each profiled strip (also referred to a profiled flat strip) has an S-shaped cross-section. The S-shaped cross-section is composed of two U-shaped longitudinal sections, wherein a first U-shaped longitudinal section is arranged distally and a second U-shaped longitudinal section is arranged proximally, each with respect to the structure, and a straight section situated between the longitudinal sections, i.e. centrally. The two U-shaped sections, which each form the longitudinal section, are bent in different directions with respect to the radial direction of the tubular structure. A first U-shaped longitudinal section is bent inwardly, whereas a second U-shaped longitudinal section is bent outwardly. The central straight section joins the two U-shaped longitudinal sections to one another via a substantially round transition. So as to ensure mobility of the mutually engaging profiled strips with respect to one another, the two U-shaped longitudinal sections are designed in such a way that one leg of the “U” is longer than the other leg. In particular, the leg that is joined to the respective other longitudinal section via the central section is longer than the other leg.

Due to the mutual engagement of the S-shaped profiled strips, in particular in the respective U-shaped longitudinal sections thereof, the profiled strips may be twisted and displaced with respect to one another, without resulting in any significant deformation of the profiled strips. The structure according to the invention thus has high flexural elasticity.

In one exemplary embodiment, the central section of an S-shaped profiled strip extends in the radial direction of the structure. As an alternative, the central section of an S-shaped profiled strip extends obliquely to the radial direction of the structure. In this way, the profile or the flexural elasticity thereof can be adapted to the respective circumstances during the implantation, i.e. the structure and the shape of the vessels through which the implant is to be displaced.

The at least one profiled strip forming the structure according to the invention preferably comprises at least one metallic material of the group consisting of steel, Co—Cr alloys, Nitinol and copper alloys and/or a stiff polymer material.

The above object is further achieved by a catheter sleeve, which is suitable, in particular, for the introduction of a stent-assisted heart valve implant, wherein the stent scaffold preferably has a self-expanding design. The catheter sleeve according to the invention comprises a stiffening sleeve and a first polymer layer, which is arranged within (i.e. in) the stiffening sleeve in the radial direction. Furthermore, a second polymer layer is provided, which is arranged outside (i.e. on the outside of) the stiffening sleeve in the radial direction, wherein the above structure according to the invention preferably forms a central section of the stiffening sleeve and/or a proximal section of the stiffening sleeve. The above-described structure according to the invention is thus accordingly arranged within the two polymer layers. In a preferred exemplary embodiment, the catheter sleeve is connected to the outer shaft of a catheter via a sleeve-shaped connector. At the distal end, the catheter sleeve can include a so-called crown made of tubular strut mesh, which widens particularly flexibly in the radial direction during the released of the implant and, if necessary, also folds again.

In an alternative embodiment of the invention, the structure according to the invention extends across the entire length of the catheter sleeve.

Due to the structure according to the invention, which forms the stiffening sleeve, the catheter sleeve according to the invention, also referred to as an implant capsule, has greater flexural elasticity and is thus able to conform better to complicated vessel shapes. Due to the displaceability of the convolutions of the tubular structure in the longitudinal direction with respect to one another, additionally high resistance to tensile and compressive stress is achieved. At the same time, the polymer layers protect the stiffening sleeve. Moreover, good mobility of the implant relative to the catheter sleeve is achieved.

The first polymer layer preferably comprises at least one material of the group consisting of Teflon and HDPE, and the second polymer layer comprises at least one material of the group comprising PEBAX. Furthermore, at least one material of the group consisting of polyolefins, polyamides, polyurethanes and polyureas, polyethers, polyesters, polyoxides, polysulfides (PPS), parax, polyether ether ketones (PEEK) and the copolymers thereof, fluorinated polymers, the aforementioned polymers mixed with barium sulfate powder and/or tungsten powder, can be used for the first and second polymer layers.

The above object is achieved analogously by a catheter comprising the above-described catheter sleeve, wherein the catheter sleeve is used and configured to receive a folded implant and is connected to the outer shaft of the catheter. The implant is preferably fixed on the inner shaft of the catheter by means of a so-called prosthesis connector. As with conventional catheters, the outer shaft is guided and movable on the inner shaft.

The catheter sleeve according to the invention is preferably part of a catheter for the insertion of a stent-assisted heart valve implant. Such a heart valve implant is composed of a valve system, preferably pericardium, which is attached to a stent scaffold and supported thereby. The stent scaffold can be balloon-expanded or preferably can be self-expanding. A self-expanding stent scaffold is preferably made of a shape memory alloy, and in particular Nitinol. Such a catheter for inserting a heart valve implant comprises at least two catheter shafts, these being an inner shaft and an outer shaft surrounding the inner shaft. The inner shaft comprises a lumen for a guide wire, and the heart valve implant is arranged on the distal region of the inner shaft. Within the scope of the present application, distal is understood to mean lying away from the treating person. The treating person accordingly has the proximal end of the catheter in his or her hand, while the distal end is situated in the body during the implantation of the heart valve implant. The outer shaft is axially displaceable with respect to the inner shaft. The distal region of the outer shaft surrounding the heart valve implant is designed as the catheter sleeve according to the invention. In the case of a self-expanding heart valve implant, the catheter sleeve holds the heart valve implant in the compressed form thereof. As a result of an axial displacement of the outer shaft with respect to the inner shaft, the self-expanding heart valve implant is at least partially no longer covered by the catheter sleeve and expands. When no part of the heart valve implant is covered any longer by the catheter sleeve, the heart valve implant is fully expanded and released.

In this embodiment, the advantages of the invention are particularly significant. The tubular structure according to the invention in the catheter sleeve according to the invention allows the heart valve implant to be reliable recaptured when it has been partially released. When the treating person establishes a malfunction or less than optimal positioning of a partially released implant, there is a need to recompress and reposition this partially released implant or to remove it from the body. For this purpose, the catheter sleeve must again be pushed completely over the partially released implant so as to bring the same into the compressed from thereof. The tubular structure according to the invention, serving as part of the catheter sleeve according to the invention, provides good axial stiffness and a high radial force, without losing pliability/flexibility.

The stiffening sleeve is preferably connected to the outer shaft 3 via a metallic connector. Such a connector ensures a good transition and good force transmission from the outer shaft onto the catheter sleeve.

Advantageously, the first polymer layer transitions, preferably seamlessly, into an inner polymer layer of the outer shaft, and the second polymer layer transitions, preferably seamlessly, into an outer polymer layer of the outer shaft, wherein particularly preferably the outer shaft comprises a metallic reinforcement between the inner and outer polymer layers. Frequently, the outer shaft also has to be reinforced. In this embodiment of the invention, the same inner polymer layer and the same outer polymer layer can be used for the catheter sleeve and the outer shaft. In particular, these can be extruded to form a workpiece when the diameters of the catheter sleeve and of the outer shaft are the same. The second polymer layer can also be applied in the form of a heat-shrinkable tube, regardless of whether the catheter sleeve and the outer shaft have identical outside diameters.

Further objectives, features, advantages, and application options of the invention will also be apparent from the following description of exemplary embodiments of the invention based on the figures. All features described and/or illustrated, either alone or in any arbitrary combination, form the subject matter of the present invention, also independently of their combination in the individual claims or their dependency reference.

Other features which are considered as characteristic for the invention are set forth in the appended claims.

Although the invention is illustrated and described herein as embodied in a structure for a catheter sleeve and a catheter sleeve, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, side perspective view of a catheter according to the invention prior to an implantation of an implant;

FIG. 2 is a perspective view of a distal section of the catheter from FIG. 1 after the implant has been released;

FIG. 3 is a side, perspective view of a catheter sleeve according to the invention comprising an outer shaft;

FIG. 4 is a cross-sectional view through the catheter sleeve according to FIG. 3 in location C (see FIG. 3);

FIG. 5 is a cross-section view of a first exemplary embodiment of a profiled strip of a structure according to the invention;

FIG. 6 is a perspective view of a second exemplary embodiment of a profiled strip of a structure according to the invention;

FIG. 7 is a longitudinal sectional view through a wall of the catheter sleeve according to the invention, comprising two mutually engaging turns of a profiled strip according to FIG. 5, in a developed view in a plane;

FIG. 8 is a longitudinal sectional view through two mutually engaging turns of the helix of a profiled strip according to FIG. 6;

FIG. 9 is a longitudinal sectional view through two mutually engaging turns of the helix of a third exemplary embodiment of a profiled strip of a structure according to the invention;

FIG. 10 is a longitudinal sectional view through two mutually engaging turns of the helix of a fourth exemplary embodiment of a profiled strip of a structure according to the invention;

FIG. 11 is a perspective view of a turn of the helix of the profiled strip according to FIG. 10;

FIG. 12 is a longitudinal sectional view through two mutually engaging turns of the helix of a fifth exemplary embodiment of a profiled strip of a structure according to the invention;

FIG. 13 is a side, perspective view of a stiffening sleeve of the catheter sleeve according to the invention, comprising a structure according to FIG. 12, in a curved state;

FIG. 14 is a sectional view of the profiled strip according to FIG. 12, in a developed view in a plane and in a view from above;

FIG. 15 is a side, perspective view of a turn of the helix of the profiled strip according to FIG. 12;

FIG. 16 is an illustration of a crown comprising a hook for attaching the crown to the catheter sleeve; and

FIG. 17 is a perspective view of an exemplary embodiment of the profiled strip of a structure according to the invention comprising a slit for receiving the hook of a crown.

### DETAILED DESCRIPTION OF THE INVENTION

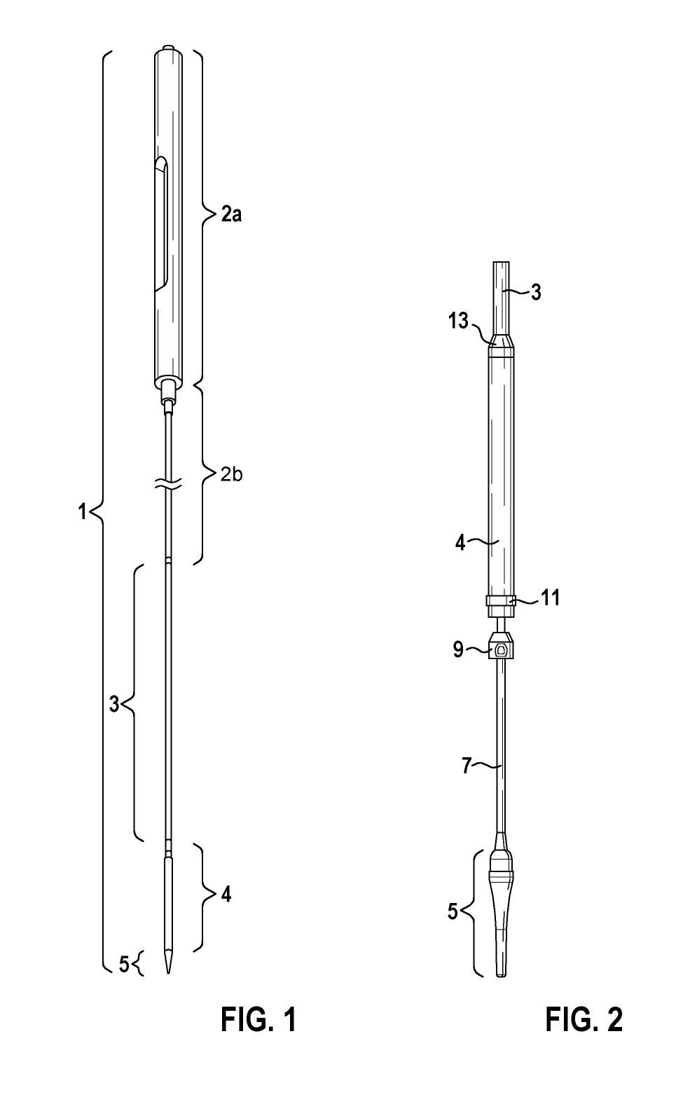

Referring now to the figures of the drawings in detail and first, particularly to FIG. 1 thereof, there is shown a catheter 1 according to the invention, comprising a handle 2a arranged at a proximal end of the catheter 1, a stabilization section 2b, an outer shaft 3, and a catheter sleeve 4 arranged on the outer shaft 3 or connected to the outer shaft 3. A dull catheter tip 5 is provided at the outermost distal end. The stabilization section 2b shields the retractable outer shaft 3 with respect to the insertion element (introducer) and the vessel wall, so that the outer shaft 3 can be freely retracted. The handle 2a is used to load, release and retract an implant arranged in the catheter sleeve 4, for example of a stent-based heart valve prosthesis. Such a stent-based heart valve prosthesis is essentially composed of a self-expanding stent scaffold, which forms the basic scaffold of the implant, and a valve system attached therein, for example made of biological tissue such as pericardium tissue. The catheter tip 5 forms the distal end of an inner shaft 7 arranged within the outer shaft 3 (see FIG. 2), wherein the catheter tip 5 is made of PEBAX, for example, mixed with barium sulfate powder or tungsten powder and visible when irradiated with X-rays.

FIG. 2 represents a distal end of the system illustrated in FIG. 1 after the implant has been released. FIG. 2 also shows that a prosthesis connector 9, by which the implant is attached to the inner shaft 7, is arranged on the inner shaft 7. The catheter sleeve 4 can comprise a ring 11 that is visible when irradiated with X-rays at the distal end to facilitate observation. The catheter sleeve 4 is connected to the outer shaft 3 by a proximal connector 13, for example.

As was already described above, in the state shown in FIG. 1 the implant is initially arranged in the catheter sleeve 4 (also referred to as a capsule) in the compressed state and is held in this state by the catheter sleeve 4. The catheter sleeve 4 is connected to the handle 2a by the outer shaft 3. In this state, the compressed implant fixed in the catheter sleeve 4 is transported through the vessels of the patient to the treatment site.

The catheter sleeve 4 is pulled toward the proximal end to release the implant. The retraction is triggered by the handle 2a and transferred onto the catheter sleeve 4 by the outer shaft 3. Initially, only a short distal section of the implant is released, and the fit is checked. If the positioning is unfavorable, the catheter sleeve can be pushed toward the distal end again by the handle 2a, whereby the implant is covered by the catheter sleeve 4 again and has transitioned completely into the compressed state. The catheter 1 can now be repositioned. Thereafter, the release of the implant arranged in the catheter sleeve 4 can start again by retraction of the outer shaft 3.

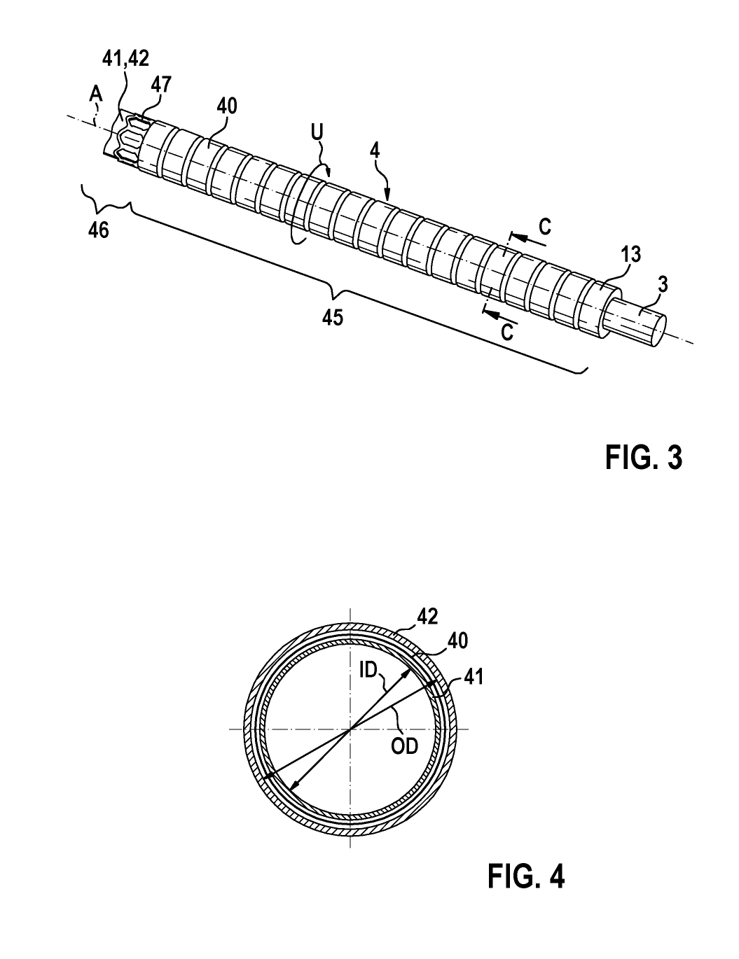

The exemplary embodiment shown in FIGS. 3 and 4 of the catheter sleeve 4 is composed of a stiffening sleeve 40, which is embedded between an inner first polymer layer 41 and an outer second polymer layer 42 surrounding the stiffening sleeve 40. The polymer layers 41, 42 surround the stiffening sleeve 40 (in other words, the polymer layers form a jacket) and protrude at the distal end thereof beyond the distal end of the stiffening sleeve 40. In the protruding regions, the polymer layers 41 and 42 are joined to one another, for example welded together or bonded to one another.

The outer second polymer layer 42 of the stiffening sleeve 40 can be made of PEBAX 7033, for example, and have a thickness of 0.04 mm. The inner first polymer layer 41 is preferably made of a low-friction material, such as Teflon or HDPE. The first polymer layer 41 can have a thickness of 0.02 mm, for example. The outside diameter OD of the catheter sleeve 4 in the region of the proximal section 45 is 5 mm to 7 mm, for example, and preferably 5.8 mm to 6.2 mm, while the inside diameter ID is 5 mm to 6 mm, for example, and preferably 5.4 mm to 5.6 mm. In any case, the inside diameter ID is smaller than the outside diameter OD.

The stiffening sleeve 40 can be divided into a proximal section 45 and a distal section 46, wherein the distal section is also referred to as a crown 46. In an alternative exemplary embodiment of the stiffening sleeve, the stiffening sleeve only comprises the proximal section 45 without the crown 46. In this alternative embodiment, the proximal section 45 covers the entire length of the heart valve implant. The proximal section 45 and the distal section 46 are arranged behind one another along the longitudinal axis/longitudinal direction A.

The stiffening sleeve 40 preferably comprises at least one metallic material of the group consisting of steel, Co—Cr alloy, Nitinol, copper alloy and/or a stiff polymer material. At the outermost proximal end of the proximal section 45, the stiffening sleeve 40 is connected to the outer shaft 3 by the proximal connector 13. A center line of the stiffening sleeve 40 forms the longitudinal direction A (see FIG. 3) of the stiffening sleeve 40 or of the catheter sleeve 4.

The distal section (the crown) 46 is formed of a strut or wire mesh, for example, which widens in a funnel shape during release of the implant so as to facilitate the release of the implant (for example, of the stent-based heart valve implant). The mesh may be joined to the proximal section 45 of the stiffening sleeve 40 by welding, for example.

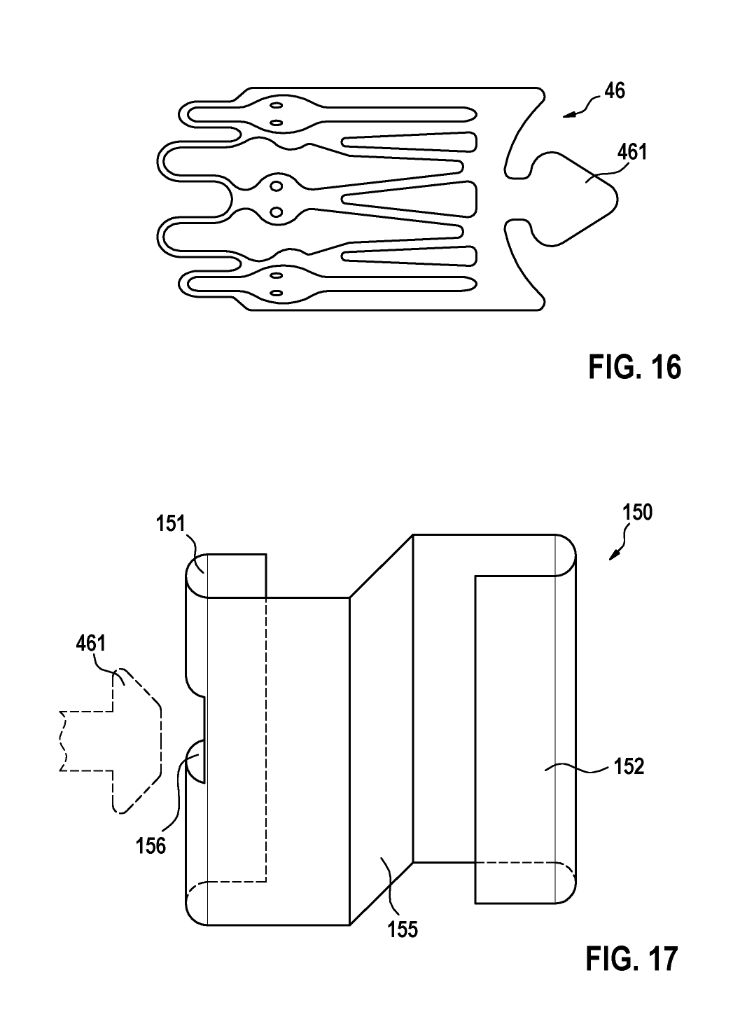

The stiffening sleeve 40 is preferably connected to the distal crown 46, wherein the connection is preferably achieved by at least one U profile, which engages in the profile of the tubular structure. In this embodiment of the invention, the existing structure of the stiffening sleeve in the catheter sleeve is utilized to connect the crown 46. As an alternative, a slot 156 is or multiple slots 156 are provided in the profile of the stiffening sleeve 40, in which one or more hooks 461 of the crown 46 can be fastened (see FIGS. 16 and 17). The crown 46 has the advantage that it can be pushed more easily over a partially released implant and thereby facilitates the resheathing of the implant.

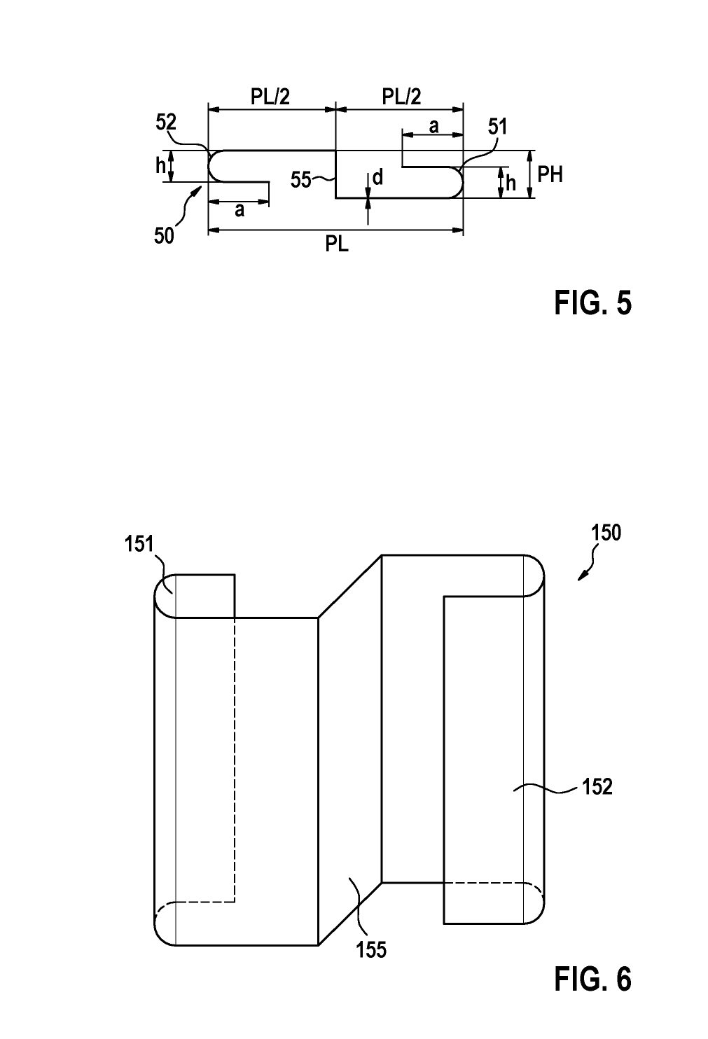

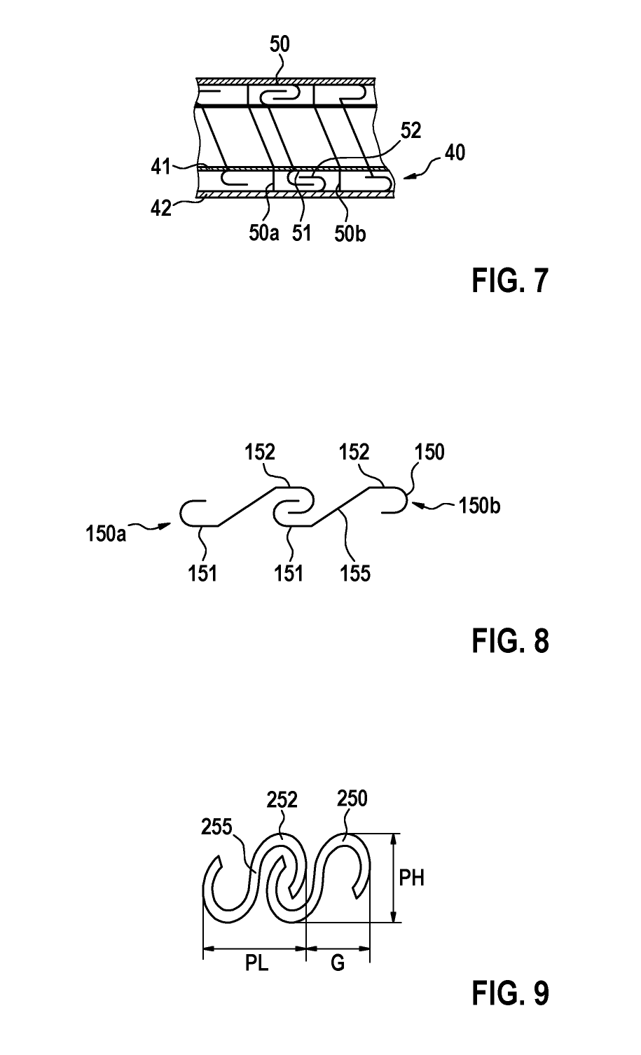

FIG. 7 shows a detail of the proximal section 45 of the stiffening sleeve 40, which is arranged in the longitudinal direction A between the distal section 46 and the connector 13. The proximal section 45 is formed by a tubular structure according to the invention made of an S-shaped profiled strip 50, which is arranged in the shape of a helix and shown in a cross-sectional view in FIG. 5. The S-shaped profiled strip 50 is wound helically along the tubular structure forming the stiffening sleeve 40 so as to form a hollow cylinder. The S-shaped profiled strip is wound with a constant slope along the circumference U of the proximal section 45 of the stiffening sleeve 40.

The profiled strip 50 has an S-shaped cross-section, which is shown in FIG. 5. Each profiled strip 50 is produced, by bending, from a flat strip having a thickness of d=0.005 mm to 0.02 mm, for example, and a width of 0.2 mm to 10 mm, for example. The length of the flat strip or profiled strip 50 can be 1000 mm, for example. The length of the profiled strip 50 is dependent on the width of the shaped profiled strip, the diameter of the stiffening sleeve 40 and the slope of the helix (or the number of convolutions). Based on the longitudinal direction A of the stiffening sleeve 40, each profiled strip 50 includes a respective U-shaped longitudinal section 51, 52 proximally and distally, which are joined by a central straight section 55. The central straight section 55 of the cross-section extends either in the radial direction based on the stiffening sleeve 40 (see FIGS. 5 and 7) or obliquely with respect to the radial direction (see FIGS. 6 and 8). In the first exemplary embodiment, the central section 55 accordingly extends perpendicularly to the legs of the U-shaped longitudinal sections 51, 52 (see FIGS. 5 and 7) or at an angle significantly different from 90° (see section 155 in FIGS. 6 and 8).

The two U-shaped longitudinal sections 51, 52 of a profiled strip 50 are bent in different directions, namely the first, distally arranged U-shaped longitudinal section 51 is bent radially to the outside and the second, proximally arranged U-shaped longitudinal section 52 is bent to the inside. The bending direction may also be reversed in each case. FIG. 7 shows the engagement of the profiled strip 50 wound helically along the circumference U of the catheter sleeve 40, wherein the section 50a forms part of a first turn of the helix and the section 50b forms part of a second turn of the helix, which adjoins the first turn. The profile has a length PL and a height PH, each of the parameters of the profiled strip 50 being shown in FIG. 5. The U-shaped longitudinal sections 51, 52 have identical dimensions, namely a short leg having the length a and a longer leg having the length PL/2 since the length of the longer leg corresponds exactly to half the total length PL/2. The height of the U-shaped longitudinal section 51, 52 is denoted by h in each case. The adjoining sections 50a, 50b corresponding to the adjoining turns of the helix engage one another in such a way that the leg of the first, outwardly bent U-shaped longitudinal section 51 is arranged in the hollow space of the second, inwardly bent U-shaped longitudinal section 52 of the first profiled strip 50a, the hollow space being formed by the two legs of the “U”. The mutual engagement of the profiled strips can take place accordingly when the bending direction of the profiled strips is reversed.

Preferred embodiments of the proximal section 45 according to the invention of the stiffening sleeve 40 have the dimensions disclosed in Table 1.

TABLE 1

| | | | | | | | Stiffening | Stiffening | | | |
|---|---|---|---|---|---|---|---|---|---|---|---|
| | | | | | | | tube | tube | Stiffening | | Min. |
| | | | | | | | outside | inside | tube | No. of | bending |
| | PL | PL/2 | a | D | h | H | diameter | diameter | length | convolutions | radius |
| V1 | 0.2 | 0.1 | 0.06 | 0.01 | 0.08 | 0.1 | 5.9 | 5.7 | 100 | 752 | 36 |
| V2 | 1 | 0.5 | 0.14 | 0.01 | 0.08 | 0.1 | 5.9 | 5.7 | 100 | 150 | 37 |
| V3 | 8 | 4 | 2.55 | 0.01 | 0.08 | 0.1 | 5.9 | 5.7 | 100 | 22 | 42 |

*) All dimensions in mm

FIG. 8 analogously shows the mutual engagement of the adjoining turns of the second exemplary embodiment of a profiled strip 150 shown in FIG. 6, comprising an obliquely extending central section 155, the profiled strip 150 otherwise being shaped similarly to the profiled strip 50 shown in FIGS. 5 and 7.

As a result of this mutual engagement, the sections 50a, 50b corresponding to turns of the profiled strip 50, 150 can be displaced toward one another in the longitudinal direction A of the catheter sleeve 4 or of the stiffening sleeve 40. This is favored, in particular, in that the leg of the U-shaped longitudinal section 51, 52, 151, 152, which is joined to the respective other U-shaped longitudinal section by means of the central section 55, 155, is longer than the respective other, opposing leg. Furthermore, it is possible to rotate with respect to one another the adjoining turns of the helix of the S-shaped profiled strip 50, 150, resulting in bending of the catheter sleeve 4 or of the stiffening sleeve 40. This results in the high flexural elasticity of the catheter sleeve 4 according to the invention in the proximal section 45 thereof.

FIGS. 9 to 15 show further exemplary embodiments of a structure according to the invention.

The third exemplary embodiment of a tubular structure according to the invention shown in FIG. 9 comprises a profiled strip 250 that, compared to the profiled strips 50 and 150 discussed above, has a clearly rounder shape and, compared to the length of the profiled strip, has a larger profile height. For example, the profile length PL=0.2 mm, the pitch of the helix G=0.133 mm, and the profile height PH=0.1 mm. The catheter sleeve, composed of the structure shown in FIG. 9, can have an outside diameter OD=5.9 mm and an inside diameter ID=5.7 mm, for example. The length of the catheter sleeve can be 100 mm, for example. For this purpose, the profiled strip 250 is placed in approximately 750 helix turns. Using such a catheter sleeve, a minimum bending radius of approximately 36 mm may be achieved, for example.

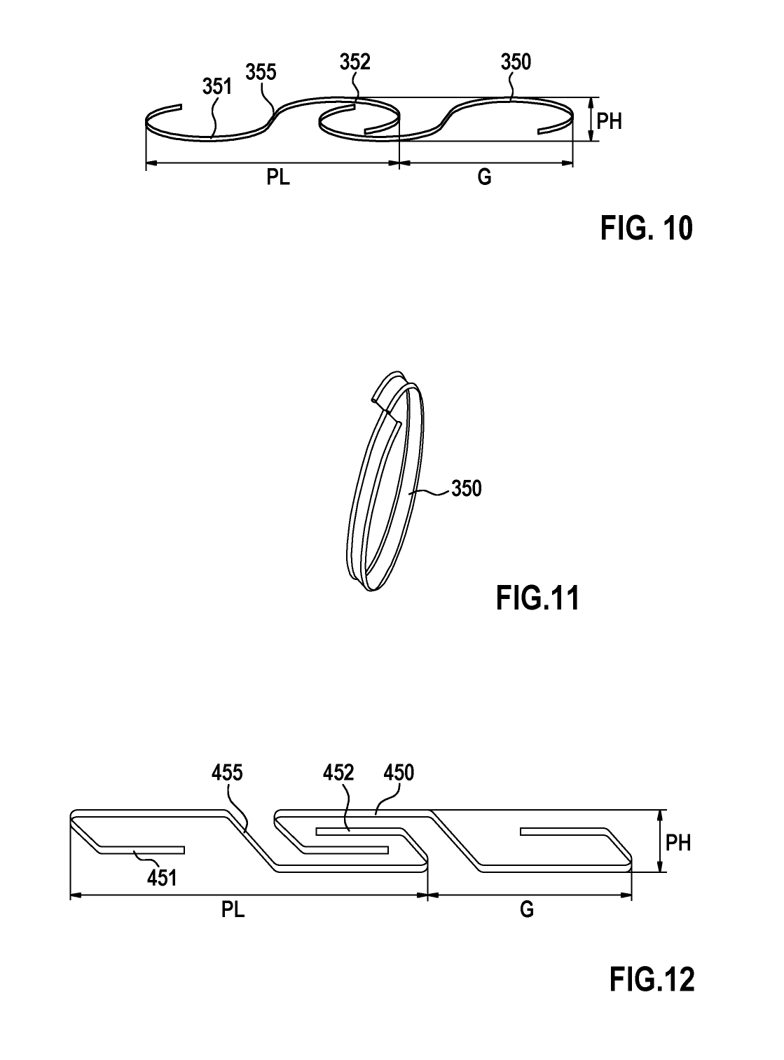

FIGS. 10 and 11 illustrate a fourth exemplary embodiment of a tubular structure according to the invention. This results from a helically wound profiled strip 350 which, compared to the exemplary embodiment shown in FIG. 9, has a considerably higher profile length PL=1 mm, compared to the profile height PH=0.1 mm. For this reason, only approximately 150 convolutions of the profiled strip 350 forming the helix are required over a length of the catheter sleeve of 100 mm. With this structure, a minimum bending radius of the catheter sleeve composed of this structure of at least 37 mm is achieved. FIG. 11 shows a convolution (turn) of the helix from the profiled strip 350 for this exemplary embodiment. Furthermore, the pitch G is shown.

The influence of the structure on the properties of the tubular structure is apparent from the exemplary embodiments of FIGS. 9, 10 and 12. Finer profiled strips 250 that are joined more closely together, as in FIG. 9, result in a considerably smaller bending radius of the tubular structure than wider profiled strips 450 joined less closely together, as in FIG. 12. The tubular structure according to FIG. 9 has a bending radius of 36 mm, the tubular structure according to FIG. 10 has a bending radius of 37 mm, and the tubular structure according to FIG. 12 has a bending radius of 42 mm. The profiled strips 450 according to FIG. 12 can be joined more easily inside one another than the profiled strips 250 according to FIG. 9, whereby the tubular structure according to FIG. 12 can be produced more easily. A person skilled in the art will thus accordingly select the optimum of bending radius and production of the respective application.

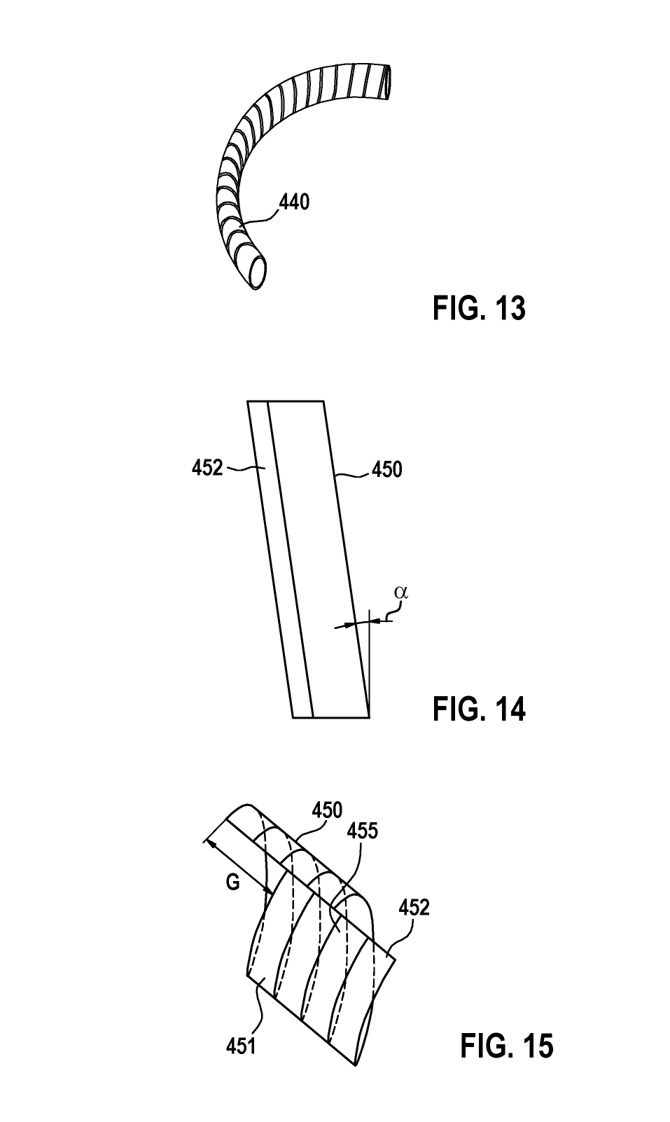

Another exemplary embodiment of a tubular structure is apparent from FIGS. 12 to 15. This structure is similar to the exemplary embodiment shown in FIGS. 5 and 8, wherein the two U-shaped longitudinal sections 451 and 452 of the profiled strip 450 have a considerably more angled shape in the cross-sectional illustration compared to the first exemplary embodiment (see FIG. 5). For example, the profile length PL=8 mm, the profile height PH=0.1 mm, and the pitch G=0.665 mm. A catheter sleeve comprising such a tubular structure has approximately 20 convolutions at a total length of the catheter sleeve of 100 mm and achieves a bending radius of the catheter sleeve of at least 42 mm. A stiffening sleeve 440 made of a structure according to this exemplary embodiment is shown in FIG. 13. Furthermore, FIG. 15 illustrates a single helix made of the profiled strip 450 having the pitch G, which is 4.9 mm, for example. In contrast, FIG. 14 shows a developed view of the profiled strip 450 having a helix angle α=15°.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

The following is a summary list of reference numerals and the corresponding structure used in the above description of the invention:
1 catheter
2a handle
2b stabilization section
3 outer shaft
4 catheter sleeve
7 inner shaft
9 prosthesis connector
11 radio-opaque ring
13 proximal connector
40 stiffening sleeve
41 first polymer layer
42 second polymer layer
45 proximal section of the stiffening sleeve 40
46 distal section of the stiffening sleeve 40
461 hooks of the crown
50, 150, 250, 350, 450 profiled strip
51, 151, 251, 351, 451 U-shaped first longitudinal section
52, 152, 252, 352, 452 U-shaped second longitudinal section
55, 155, 255, 355, 455 central section
156 slit
A longitudinal direction (longitudinal axis) of the catheter sleeve 4
ID inside diameter of the catheter sleeve 4
OD outside diameter of the catheter sleeve 4
U circumferential direction of the catheter sleeve 4
a length of the shorter leg of the U-shaped longitudinal section 51, 52
PL/2 length of the longer leg of the U-shaped longitudinal section 51, 52
d thickness of the profiled strip 50
h height of the U-shaped longitudinal section 51, 52
PH total height of the profile of the respective profiled strip in the cross-section
PL total length of the profile of the respective profiled strip in the cross-section
G pitch of the helix

1. A tubular structure for a catheter sleeve, the tubular structure comprising:
at least one S-shaped profiled strip being disposed along the tubular structure in a helix, said at least one S-shaped profiled strip having a U-shaped longitudinal section in a region of each side edge, a respective said U-shaped longitudinal section of said at least one S-shaped profiled strip engaging with an opposing, U-shaped longitudinal section of a directly adjoining turn of said helix of said at least one S-shaped profiled strip or of a further S-shaped profiled strip adjoining in said helix.

2. The structure according to claim 1, wherein said at least one S-shaped profiled strip has a central section which in cross-section extends either in a radial direction or obliquely with respect to the radial direction.

3. The structure according to claim 1, wherein said helix is right-handed or left-handed.

4. The structure according to claim 2, wherein said U-shaped longitudinal section has a first leg joined directly to said central section and a second leg, said first leg is longer than said second leg

5. The structure according to claim 1, wherein said at least one S-shaped profiled strip contains at least one metallic material selected from the group consisting of steel, Co—Cr alloys, Nitinol and copper alloys and a stiff polymer material.

6. A catheter sleeve, comprising:
a stiffening sleeve containing a tubular structure having at least one S-shaped profiled strip being disposed along said tubular structure in a helix, said at least one S-shaped profiled strips having a U-shaped longitudinal section in a region of each side edge, a respective said U-shaped longitudinal section of said at least one S-shaped profiled strip engaging with an opposing, U-shaped longitudinal section of a directly adjoining turn of said helix of said at least one S-shaped profiled strip or of a further S-shaped profiled strip adjoining in said helix, said tubular structure defining a central and/or proximal section of said stiffening sleeve;
a first polymer layer, said first polymer layer in a radial direction is disposed within said stiffening sleeve; and
a second polymer layer, said second polymer layer in the radial direction is disposed outside said stiffening sleeve.

7. A catheter sleeve, comprising:
a stiffening sleeve containing a tubular structure having at least one S-shaped profiled strip being disposed along said tubular structure in a helix, said at least one S-shaped profiled strip having a U-shaped longitudinal section in a region of each side edge, a respective said U-shaped longitudinal section of said at least one S-shaped profiled strip engaging with an opposing, U-shaped longitudinal section of a directly adjoining turn of said helix of said at least one S-shaped profiled strip or of a further S-shaped profiled strip adjoining in said helix, said tubular structure formed over an entire length of said stiffening sleeve;
a first polymer layer, said first polymer layer in a radial direction is disposed within said stiffening sleeve; and
a second polymer layer, said second polymer layer in the radial direction is disposed outside said stiffening sleeve.

8. A catheter, comprising:
an outer sleeve;
a catheter sleeve according to claim 6, wherein said catheter sleeve is connected to said outer shaft and configured to receive an implant.

9. The catheter according to claim 8, further comprising a metallic connector, said stiffening sleeve is connected to said outer shaft by means of said metallic connector.

10. The catheter according to claim 8, wherein:
said outer shaft has an inner polymer layer and an outer polymer layer;
said first polymer layer transitions into said inner polymer layer; and
said second polymer layer transitions into said outer polymer layer.

11. The catheter according to claim 8, further comprising a distal crown connected to said stiffening sleeve.

12. The catheter according to claim 11, wherein said distal crown has at least one U profile which engages in a profile of said tubular structure.

13. The catheter according to claim 11, wherein:
said tubular structure has a slit formed therein; and
said distal crown has a least one hook engaging in said slit of said tubular structure.

14. The catheter according to claim 10, wherein said outer shaft has a metallic reinforcement between said inner polymer layer and said outer polymer layer.
